(19) United States
(12) Patent Application Publication
(10) Pub. No.: US 2015/0289828 A1
(43) Pub. Date: Oct. 15, 2015

(54) PATIENT TABLE WITH INTEGRATED X-RAY VOLUMETRIC IMAGER

(71) Applicant: L-3 Communications Security and Detection Systems, Inc., Woburn, MA (US)

(72) Inventors: Andrew D. Foland, Wellesley, MA (US); Michael H. Schmitt, Woburn, MA (US)

(73) Assignee: L-3 Communications Security and Detection Systems, Inc., Woburn, MA (US)

(21) Appl. No.: 14/682,192

(22) Filed: Apr. 9, 2015

Related U.S. Application Data
(60) Provisional application No. 61/977,745, filed on Apr. 10, 2014.

(51) Int. Cl. A61B 6/00; G06T 15/08; G06T 11/00; A61B 6/04; A61B 6/10

(52) U.S. Cl. CPC A61B 6/4014; A61B 6/0407; A61B 6/52; A61B 6/107; G06T 11/008; G06T 15/08; G06T 2207/10081

(57) ABSTRACT

Methods and apparatus for integrating a table with at least one X-ray source for medical imaging of patients. The apparatus comprises a table on which a patient may be placed, at least one X-ray source configured to generate X-rays at a plurality of X-ray source locations along a linear direction, wherein the at least one X-ray source is arranged to generate the X-rays such that at least some of the X-rays pass through a portion of the table in addition to passing through a portion of a patient placed on the table, and at least one detector array comprising a plurality of detector elements and arranged to detect the at least some of the X-rays passed through the portion of the patient placed on the table, wherein the at least one detector array comprises detector elements arranged in a two-dimensional configuration. Iterative reconstruction techniques may be used to reconstruct an image from X-ray data detected using the at least one detector.

### CROSS-REFERENCE TO RELATED APPLICATIONS

This Application claims the benefit under 35 U.S.C. §119(e) of U.S. Provisional Application Serial No. 61/977,745 entitled “Patient Table with Integrated X-Ray Volumetric Imager” filed Apr. 10, 2014, under Attorney Docket No. L0632.70116US00, which is herein incorporated by reference in its entirety.

### BACKGROUND

X-ray imaging technology has been employed in a wide range of applications from medical imaging to detection of unauthorized objects or materials in baggage, cargo or other containers generally opaque to the human eye. X-ray imaging typically includes passing high-energy radiation (i.e., X-rays) through an object to be imaged. X-rays from a source passing through the object interact with the internal structures of the object and are altered according to various characteristics of the material (e.g., transmission, scattering and diffraction characteristics, etc.). By measuring changes (e.g., attenuation) in the X-ray radiation that exits the object, information related to material through which the radiation passed may be obtained to form an image of the object.

In order to measure X-ray radiation penetrating an object to be imaged, an array of detectors responsive to X-ray radiation typically is arranged on one side of the object opposite a radiation source. The magnitude of the radiation, measured by any detector in the array, represents the density of material along a ray from the X-ray source to the X-ray detector. Measurements for multiple such rays passing through generally parallel planes through the object can be grouped into a projection image. Each such measurement represents a data point, or “pixel,” in the projection image.

Projection imaging is well suited for finding objects that have material properties or other characteristics such that they produce a group of pixels having a recognizable outline regardless of the orientation of the object to be imaged. However, projection images are not well suited for reliably detecting or characterizing some objects. If the rays of radiation pass through only a thin portion of the object or pass through multiple objects, there may be no group of pixels in the projection image that has characteristics significantly different from other pixels in the image. The object may not be well characterized by, or even be detected in, the resultant projection image.

Measuring attenuation of X-rays passing through an object from multiple different directions can provide more accurate detection of relatively thin objects. For instance, in a computed tomography (CT) scanner, such measurements may be obtained by placing the X-ray source and detectors on a rotating gantry. As the gantry rotates around the object, measurements are made on rays of radiation passing through the object from many different directions.

Multiple projection images can be used to construct a three-dimensional, or volumetric, image of the object. A volumetric image is organized in three-dimensional sub-blocks called “voxels”—analogous to pixels in a two-dimensional image—with each voxel corresponding to a density (or other material property) value of the object at a location in three-dimensional space. Even relatively thin objects may form a recognizable group of voxels in such a volumetric image.

The process of using multiple radiation measurements from different angles through an object to compute a volumetric image of the object is herein referred to as volumetric image reconstruction. The quality of volumetric image reconstruction not only depends on the geometry of the imaged object, but also on the geometry of the imaging system including the relative positions of X-ray sources and detectors used to make the measurements. The relative positions of sources and detectors control the set of angles from which each voxel is irradiated by X-rays.

CT scanners have also found utility for medical applications where a portion of a patient may be scanned to determine the extent of an injury or other medical condition. For example, a patient may be scanned using a CT scanner prior to undergoing surgery to remove implanted foreign objects as a result of an automobile accident, an explosion, or some other traumatic event. Reconstructing a volumetric image of the portion of the patient that will be operated on provides the surgeon with information about the foreign object(s) to help guide the surgical intervention.

### SUMMARY

The inventors have recognized and appreciated that some conventional X-ray scanners have limited application in environments where rapid CT scans of patients would facilitate medical intervention. For example, in some military applications, it would be advantageous to perform CT scans on wounded soldiers on the battlefield to quickly assess injuries prior to performing surgery on such patients. However, some conventional CT scanners are large machines that are not portable or easily transportable by a vehicle (e.g., a helicopter) to battlefields or other similar environments. Additionally, conventional CT scanners, which typically include X-ray sources and/or detectors located on a rotating gantry, include moving parts that may not perform well in harsh physical environments with varying degrees of temperature fluctuation and other physical impediments. Accordingly, some embodiments are directed to methods and apparatus for rapidly obtaining X-ray images of a patient using a portable X-ray imager integrated with a table (e.g., an operating table, a surgical table, a stretcher, a litter, a gurney, etc.).

Although illustrative embodiments described in more detail below relate to deployment of embodiments for military applications (e.g., on battlefields), it should be appreciated that embodiments of the invention are not restricted based on any particular application. For example, some embodiments may be used in emergency applications (e.g., on an ambulance, for search and rescue), in conventional medical facility environments (e.g., a surgical room of a hospital), or embodiments may be used for any other suitable application or with any other suitable environment.

In one aspect, some embodiments are directed to an apparatus, comprising: a table on which a patient may be placed; at least one X-ray source configured to generate X-rays at a plurality of X-ray source locations along a linear direction, wherein the at least one X-ray source is arranged to generate the X-rays such that at least some of the X-rays pass through a portion of the table in addition to passing through a portion of a patient placed on the table; and at least one detector array comprising a plurality of detector elements and arranged to detect the at least some of the X-rays passed through the portion of the patient placed on the table.

In another aspect, some embodiments are directed to a method of manufacturing an apparatus comprising a table on which a patient may be placed; at least one X-ray source configured to generate X-rays at a plurality of X-ray source locations along a linear direction, wherein the at least one X-ray source is arranged to generate the X-rays such that at least some of the X-rays pass through a portion of the table in addition to passing through a portion of a patient placed on the table; and at least one detector array comprising a plurality of detector elements and arranged to detect the at least some of the X-rays passed through the portion of the patient placed on the table.

In another aspect, some embodiments are directed to a method of volumetric image reconstruction comprising: receiving X-ray data from at least one detector array, wherein the received X-ray data does not satisfy a volumetric reconstruction requirement; and reconstructing, with at least one computer processor, the volumetric image using an iterative reconstruction technique based, at least in part, on the received data.

In another aspect, some embodiments are directed to a non-transitory computer readable medium encoded with a plurality of instructions that, when executed by at least one computer processor perform a method. The method comprises receiving X-ray data from at least one detector array, wherein the received X-ray data does not satisfy a volumetric reconstruction requirement; and reconstructing, with the at least one computer processor, the volumetric image using an iterative reconstruction technique based, at least in part, on the received data.

It should be appreciated that all combinations of the foregoing concepts and additional concepts discussed in greater detail below (provided that such concepts are not mutually inconsistent) are contemplated as being part of the inventive subject matter disclosed herein.

### BRIEF DESCRIPTION OF THE DRAWINGS

FIG. 1 is schematic of an illustrative imaging apparatus in accordance with some embodiments;

FIG. 2 is a sketch illustrating aspects of forming a multiview volumetric image, in accordance with some embodiments;

FIG. 3 is a schematic of an illustrative surgical table that may be used in accordance with some embodiments; and

FIG. 4 is an illustrative process for reconstructing a volumetric image in accordance with some embodiments.

### DETAILED DESCRIPTION

Challenging environmental conditions, such as military battlefields or forward medical stations provide unique challenges that are not well suited for imaging injured patients using conventional CT scanners. For example, it is very difficult to obtain timely CT scans for diagnosis or surgical guidance on injured soldiers in emergency situations. Accordingly, some embodiments of the invention are directed to rapid-acquisition, in-situ, field-robust, portable X-ray imaging methods and apparatuses that are practical for use in such challenging environments.

Some embodiments of the invention are directed to an X-ray apparatus integrated with a table on which a patient may be placed. The table may be a surgical table on which the patient is placed for a surgical procedure, such as removal of one or more bullets from a gunshot wound, removal of one or more pieces of shrapnel from a battlefield injury, etc. A limitation of some conventional CT scanners is that they may not be closely integrated with surgical tables, such that repeated imaging of a patient cannot be performed during a surgical procedure without substantial adjustment of the CT scanner and/or the patient between each imaging session. For example, conventional tomosynthetic C-arm scanners typically require repositioning and/or repositioning of the patient prior to the imaging session and following the imaging session to continue the surgical procedure. Additionally, conventional CT scanners when used during a surgical procedure, typically obstruct a surgeon or other medical professional from accessing the patient on the table during the imaging session. Accordingly, some embodiments are directed to an X-ray apparatus integrated with a surgical table that does not obstruct a physician from accessing a patient during and/or immediately following an imaging session. Other advantages of embodiments of the invention are apparent from the discussion following below.

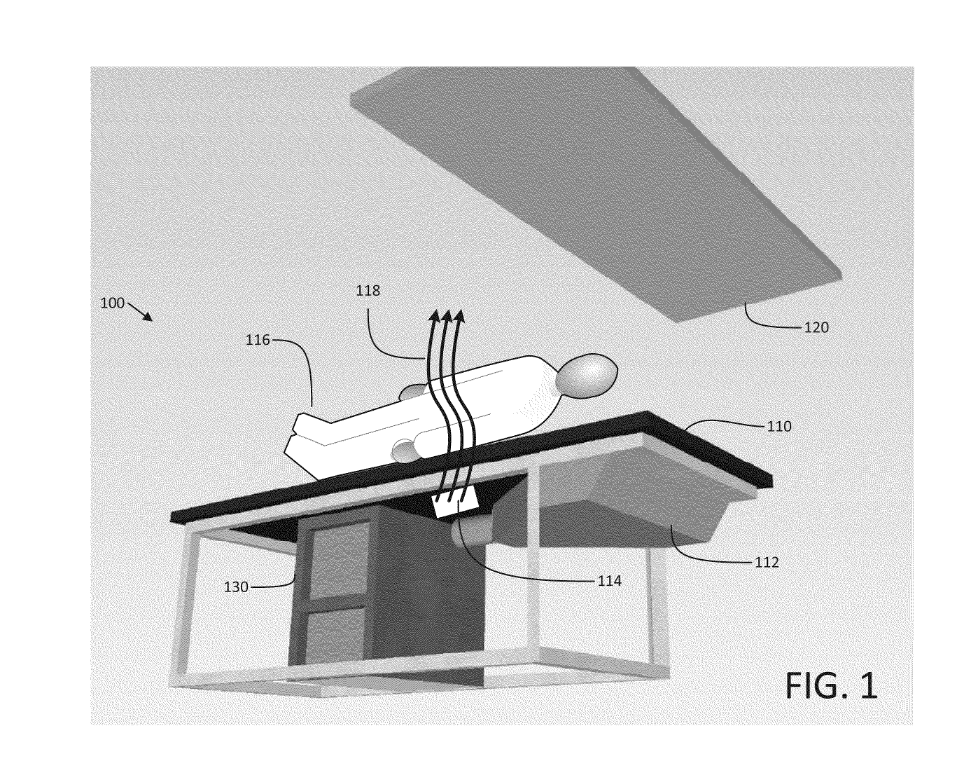

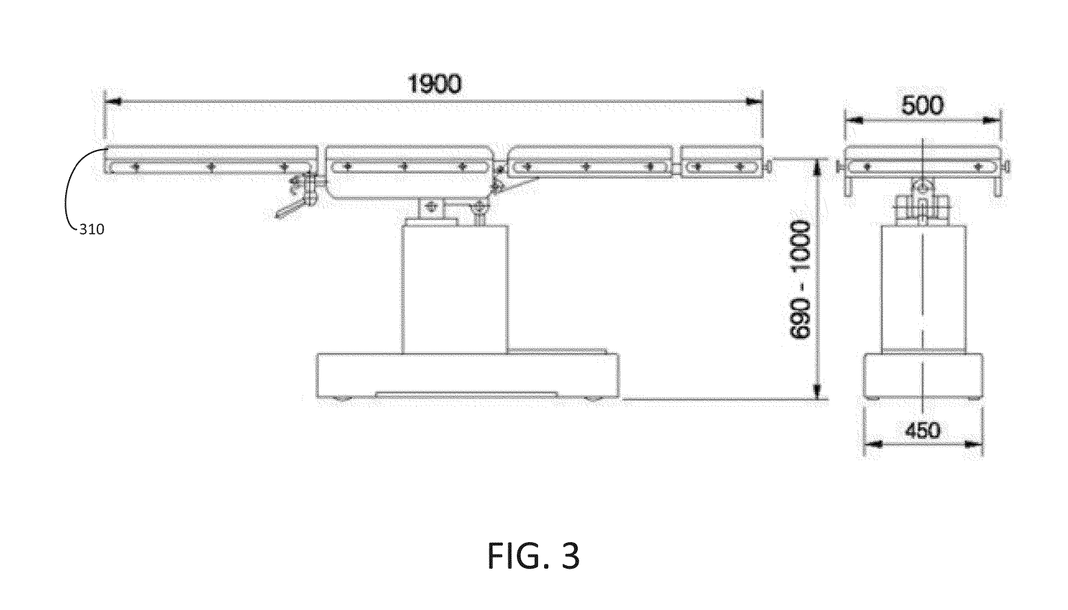

FIG. 1 shows an imaging apparatus 100 in accordance with some embodiments of the invention. Imaging apparatus 100 includes table 110 on which a patient 116 may be placed. In some embodiments table 110 may be a surgical table having standard dimensions of 76″×20″×30″ or any other suitable dimensions. FIG. 3 shows a schematic of an illustrative surgical table 310 with exemplary dimensions shown in millimeters.

Table 110 may be made of any suitable material including, but not limited to, steel, carbon fiber, fabric, cloth, canvas, and wood. At least a portion of table 110 may be made of a material that enables X-rays to pass through the at least a portion of the table. For example, in some embodiments, at least a portion of table 110 may comprise carbon fiber material that forms a window 114 through which X-rays generated by at least one X-ray source may pass. In some embodiments, table 110 may be implemented as a removable litter, gurney, or stretcher, and the at least one window 114 in table 110 may comprise canvas, fabric, cloth, wood, or any other suitable material that enables X-rays to pass through the at least one window. In some embodiments, at least one window in the X-ray table may be devoid of material such that X-rays generated by at least one X-ray source may pass through the at least one window that does not include any material. The at least one window in table 110 may be any suitable size including, but not limited to, the size of the entire table surface.

In some embodiments, at least a portion of table 110 may be movable such that the at least a portion of the table may translate in one or more directions to enable different portions of a patient placed on the table to be in the path of X-rays generated by at least one X-ray source. Translation of table 110 may achieved in any suitable way. For example, some embodiments may include one or more rails that enable at least a portion of the table to be translated in the length direction and/or the width direction of the table. In other embodiments, table 110 may be stationary, and at least one X-ray source used to image a patient may be movable, for example, along one or more rails attached to the table.

Imaging apparatus 100 also includes X-ray source 112 configured to generate X-ray radiation 118 that passes through at least a portion of table 110. In some embodiments, X-ray source 112 is a linear X-ray source configured to generate X-rays at a plurality of X-ray source locations along a linear direction. In some embodiments, X-ray source 112 is a stationary X-ray source that generates X-ray radiation at a series of time-multiplexed spatial locations passing through table 110 without requiring any moving parts (e.g., without requiring a rotatable gantry). A stationary source, as that term is used herein, is a source that does not move during a single acquisition of an image. Such stationary sources may be electronically-controlled, such that X-ray energy may be generated at different spatial locations. An example of such a stationary X-ray source is an e-beam. In e-beam imaging systems, one or more e-beams are directed to impinge on the surface of a target responsive to the e-beams. The target may be formed from, for example, tungsten, molybdenum, gold, or other material that emits X-rays in response to an electron beam impinging on its surface. For example, the target may be a material that converts energy in the e-beam into X-ray photons, emitted from the target essentially in the 4π directions. The released energy may be shaped or collimated by blocking selected portions of the X-rays emitted from the target using any of various radiation absorbing material (such as lead). For example, the X-ray may be collimated to form a cone beam, a fan beam, a pencil beam or any other X-ray beam having generally desired characteristics. The collimated X-rays may then pass into an inspection region to penetrate an object of interest to ascertain one or more characteristics of the object.

While conventional X-ray scanning systems employ one or more sources and detectors positioned or rotated in a circular geometry, e-beam imaging systems may comprise arbitrary, and more particularly, non-circular geometries, which offers a number of benefits with respect to the flexibility of the design and may facilitate more compact and inexpensive X-ray detection system. In an exemplary X-ray scanning system, the target which converts energy in an e-beam to X-ray energy may be provided as one or more linear segments.

Additionally, different types of stationary sources may be used in various embodiments of the invention. For example, in one implementation, X-ray source 112 may comprise a plurality of carbon nanotube elements that each act as an individual source activated by applying in time-sequence a signal to each of the elements. An X-ray source comprising a plurality of carbon nanotube elements may also be configured as a linear source in accordance with the techniques described herein.

In other embodiments, X-ray source 112 may comprise a distributed array of switchable X-ray sources that, when activated in time-sequence, emit X-ray radiation. The switchable X-ray sources in the distributed array may be activated by application of any suitable signal to each source including, but not limited to, a voltage and a light source.

In other embodiments, X-ray source 112 may comprise a multi-energy X-ray source that emits X-ray radiation at more than one energy level. For example, the inspection system may include one or more X-ray generation subsystems adapted to generate X-ray radiation at a first energy level and a second energy level. Alternatively, a multi-energy X-ray source may emit X-ray radiation at more than two energy levels. To support multi-energy imaging, each X-ray generation subsystem may generate radiation of a different energy level during successive intervals when it operates. By correlating detector outputs to times in which the X-ray generation subsystems are generating, for example, high-and low-energy X-rays, high and low X-ray data may be collected for a multi-energy image analysis. Such an analysis may be performed using techniques as known in the art or in any other suitable way.

X-ray source 112 may be integrated with table 110 in any suitable way. In some embodiments, X-ray source 112 may be mounted substantially below table 110. In such a configuration, X-rays generated from the X-ray source pass upward through table 110 and are detected by at least one detector array 120 mounted above table 110, as discussed in further detail below. In some embodiments, X-ray source may be mounted entirely below table 110, as shown in FIG. 1. In other embodiments, at least a portion of X-ray source 112 may extend at least partially above table 110. For example, X-ray source 112 may include an portion that enables X-rays to be generated in a direction perpendicular to the bottom surface of table 110 (i.e., along the length direction and/or the width dimension of the table). In some embodiments, the portion of X-ray source 112 extending above table 110 may be adjustable such that in a first configuration the X-ray source is disposed entirely below the table and in a second configuration the portion of the X-ray source is extended above the table. In embodiments in which at least a portion of X-ray source 112 is mounted below table 110, at least some X-rays generated by X-ray source 112 pass through at least a portion of the table prior to passing through a patient placed on the table.

As discussed above, in some embodiments X-ray source 112 may be movable relative to at least a portion of table 110 to enable different portions of a patient placed on table 110 to be imaged without moving the patient on the table. Any suitable mechanism may be used to enable X-ray source 112 to be translated along the length dimension and/or the width dimension of table 110 including, but not limited to, using one or more rails on which the X-ray source 112 and/or at least a portion of table 110 may move.

Although X-ray source 112 is shown as being mounted below table 110, it should be appreciated that in some embodiments, X-ray source 112 may alternatively be mounted above table 110 and configured to generate X-ray radiation downward through the top surface of table 110. In such an embodiment, at least one detector array may be integrated as a portion of the table 110 or mounted below table 110 to detect radiation passing through the table.

Imaging apparatus 100 also includes detector array 120 comprising a plurality of detector elements and arranged to detect X-rays passed through the portion of the table 110 from X-ray source 112. Detector array 120 may include any suitable type of detectors for detecting X-rays, and the detectors may be arranged in any suitable two-dimensional configuration. In some embodiments, detector array 120 comprises a flat panel detector array, as shown in FIG. 1. In some embodiments, detector array 120 may comprise a plurality of linear arrays of detectors arranged in a two-dimensional configuration. In other embodiments, detector array 120 comprises a photodiode array with scintillator elements. In some embodiments, detector array 120 may include one or more detector arrays mounted to a movable structure. Mounting the detector array 120 to a movable structure may enable the detector array to be translated over a patient placed on the table to image different parts of the body. Alternatively, mounting the detector array 120 to a movable structure may enable the detector array to be moved out of the way during a medical procedure.

In embodiments employing a multi-energy X-ray source, at least some of the detectors in detector array 120 may be configured to classify received X-ray radiation as having one of a plurality of energies, such as a first energy or a second energy. For example, some or all of the detectors in detector array 120 may be adapted to record individual X-ray photon arrival energies with sufficient resolution to separate photons having a first energy from photons having a second energy. The detectors may be configured to classify the energy of received X-ray radiation by, for example, being constructed of a material, such as CdZnTe (CZT) that enables the classification of individual photons. Such detectors are known in the art and are often commonly referred to as photon-counting detectors or multispectral detectors.

In some embodiments, detector array 120 may be mounted above table 110 as shown in FIG. 1. Detector array 120 may be mounted in any suitable way including, but not limited to, mounting the detector array to a vehicle such as a land, air, or sea-based military vehicle, a helicopter, an ambulance, or an airplane. For example in some embodiments detector array 120 is mounted to a military vehicle, such as a sea vessel that cannot accommodate a conventional CT systems due to physical size constraints of the ship/sea vessel and the form factor of the CT system (e.g., conventional CT systems may not fit through the hatches in such vessels).

As discussed above, some embodiments enable an integrated medical X-ray scanner to be portable, such that the X-ray scanner can be transported for military and/or emergency applications for which conventional X-ray scanners are incompatible. Some embodiments are of such a weight that they are helicopter-transportable. For example, imaging apparatus manufactured in accordance with some embodiments may be less than ten thousand pounds, less than eight thousand pounds, less than five thousand pounds, or less than two thousand pounds. Other embodiments are of a size that they are transportable in a vehicle such as an ambulance. To achieve such a size, some embodiments may include a compact X-ray source configured to fit entirely or substantially entirely within the dimensions of a conventional size ambulance gurney or table.

Conventional CT systems having a large contiguous structure, where several components of the CT system including the X-ray source and the detector array, are mounted on rotating gantry, have limited portability and configurability due to their size and form factor. In some embodiments, one or more components of the X-ray scanner are provided (e.g., manufactured) as modules that may be separately transported to a location where the X-ray scanner is to be assembled, and the modular pieces of the system may be assembled at the desired location. For example, in some embodiments, one or more of an X-ray source, a detector array, a power source, and other electronics of the X-ray system may be provided as separate modules that may be assembled into a an X-ray system for generating X-ray-based images (e.g., CT images). The modularity of such embodiments contributes to the portability of the X-ray system

Rather than being deployed in military or emergency vehicles or vessels, X-ray scanners in accordance with some embodiments may be installed in traditional medical facilities such as hospitals. In such applications, detector array 120 may be mounted to the ceiling of an operating room or other room at the medical facility. When mounted to the ceiling, detector array 120 may be fixed to the ceiling or mounted on a movable structure that can be brought closer to the patient during imaging. Mounting detector array 120 on a movable structure may enable the detector array to be reduced in size compared to mounting the detector array to the ceiling in a fixed configuration. In some embodiments, detector array 120 may alternatively be mounted on a movable or fixed structure rather than being mounted on the ceiling of a vehicle or structure.

In some embodiments, detector array 120 is associated with read-out circuitry configured to read out information from the detector elements of the detector array. The read out circuitry may be configured to simultaneously read out information from all detector elements of the detector array or a subset (i.e., less than all) of the detector elements of the detector array. Information read out from the detector elements of the detector array may be provided to at least one computer to perform a volumetric image reconstruction based on the read out information, as discussed in more detail below.

Imaging apparatus 100 also includes a computer 130 including at least one processor programmed to reconstruct a volumetric image based, at least in part, on X-rays detected by detector array 120. In some embodiments, computer 130 may be integrated with imaging apparatus 100 as shown in FIG. 1. In other embodiments, computer 130 may be located remote from imaging apparatus 100 and X-ray data output from detector array 120 may be transmitted to the remotely-located computer 130 for image reconstruction and/or analysis. For example, in a military application where a patient is imaged on the front lines of a battlefield by imaging apparatus 100, X-ray data output from detector array 120 may be transmitted to a computer 130 located in a safer location where a physician can analyze the images being reconstructed based on the collected detector data. Alternatively, when computer 130 is integrated with imaging apparatus 100, the image can be reconstructed using computer 130, and the reconstructed image may be transmitted to a remotely-located computer for analysis, as embodiments of the invention are not limited in the particular arrangement or location of computer 130.

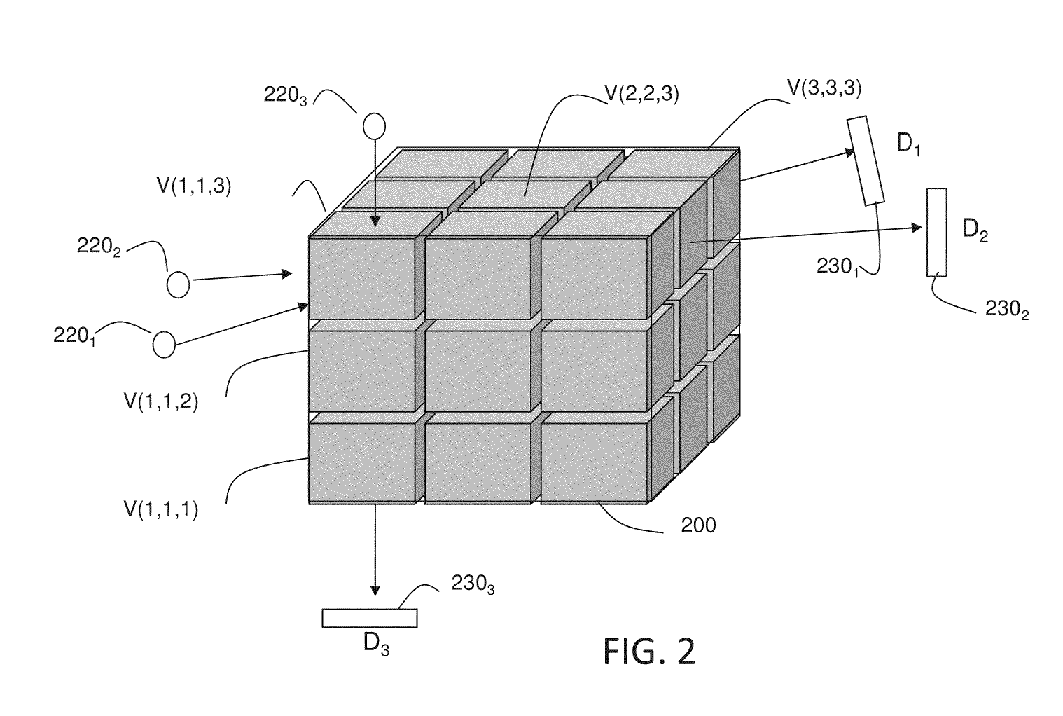

FIG. 2 is a sketch demonstrating aspects of computing a volumetric image from measurements made on an object 200 (e.g., a region of the patient's body). In the simple example of FIG. 2, the imaged object 200 is divided into nine regions. An image of the object 200 is formed by computing a property of the material in each of these nine regions. Each of the nine regions will correspond to a voxel in the computed image. For this reason the regions in the object are sometimes also referred to as “voxels.” In the simple example of FIG. 2, object 200 is divided into nine voxels of which V(1,1,1), V(1,1,2), V(1,1,3), V(2,2,3) and V(3,3,3) are numbered. To form a volumetric image of object 200, a material property is computed for each of the voxels from the measured outputs of detectors, of which detectors 230₁, 230₂ and 230₃ are shown. In the illustrated embodiment, the material property is an average density of the material within the voxel.

In the embodiment illustrated, measurements from which density may be computed are made by passing rays of radiation through the object 200 from different directions. By measuring the intensity of the rays after they have passed through the object and comparing the measured intensity to incident intensity, attenuation along the path of the ray may be determined. If attenuation along a sufficient number of rays traveling in a sufficient number of directions is measured, the data collected can be processed to compute the density within each of the voxels individually.

For example, FIG. 2 shows a source 220₁ and a detector 230₁. A ray traveling from source 220₁ to detector 230₁ passes through voxels V(1,1,3), V(2,2,3) and V(3,3,3). As a result, the value measured at detector 230₁ will depend on the densities in each of those voxels. Thus, the measurement taken at detector 230₁ of a ray from source 220₁ may be used to estimate the density at each of the voxels V(1,1,3), V(2,2,3) and V(3,3,3).

As shown, a ray from source 220₁ to detector 230₁ represents just one of the rays passing through object 200. Other rays are shown in the example of FIG. 2. For example, a ray is shown passing from source 220₂ to detector 230₂. As with the ray passing from source 220₁ to detector 230₁, the value measured at detector 230₂ will depend on the densities of voxels V(1,1,3), V(2,2,3) and V(3,2,3) because the ray source 220₂ passes through these voxels before impinging on detector 230₂. Similarly, the value measured at detector 230₃, with respect to a ray passing from passing from source 220₃ to detector 230₃, is influenced by the densities of the voxels along that ray (V(1,1,1), V(1,1,2), and V(1,1,3)).

FIG. 2 shows only three rays passing through object 200. Each of the rays generates a single measurement representative of the densities of voxels, through which the ray passes, in object 200. In the simple problem illustrated in FIG. 2, object 200 is divided into 27 voxels. Accordingly, though FIG. 2 shows only three rays passing through object 200, to compute a volumetric image of object 200, more measurements are typically needed.

In a physical system, the number of measurements taken often exceeds the number of voxels in the image. For instance, measurements may be made such that multiple rays pass through each voxel with some of the rays passing through each voxel from a range of angles. The range of angles may be any suitable range. For example, it may be desirable to have rays passing through the object from a range of angles that exceeds 180°, or a range of angles that is as close to 180° as possible. Though in other scenarios the range of angles may be smaller, for instance a range such as 170°, 160°, 150°, or 140°, or even less may be used.

The inventors have recognized and appreciated that in certain implementations (e.g., military and portable emergency implementations) acquiring rapid images may be as important or more important than obtaining high quality images. Accordingly, in some embodiments, a volumetric image is reconstructed that includes some imaging artifacts. For example, the volumetric image may be reconstructed using information that does not satisfy one or more volumetric reconstruction requirement. Any suitable volumetric reconstruction requirement may be used including, but not limited to a Tuy condition, a pi-line-condition, a Nyquist condition, and a non-truncation condition. Additionally, in some embodiments a volumetric imaging reconstruction may be performed using information corresponding to a range of angles substantially less than 180°. In some embodiments, a controller may be provided to control operation of the X-ray source(s) to achieve any desired range of angles including a range of angles less than 180°.

Measurements obtained from multiple rays passing through the object under inspection may be used to compute a volumetric image. For instance, if a sufficient number of measurements along rays from a sufficient number of independent angles are made, the measured outputs of the detectors may be used to define a system of simultaneous equations that, using an iterative mathematical technique, may be solved for the unknown values representing the densities of the individual voxels in object 200.

Uncertainty or other variations in the measurement process may prevent a single solution from satisfying simultaneously all equations in a system of equations formed from the measurements. Thus, solving the system of equations formed from actual measurements would involve finding the values that best solve the equations. Similarly, obtaining measurements from multiple angles will allow voxels to be computed using a direct method.

In some embodiments, an iterative reconstruction technique is used to reconstruct a volumetric image of an object. Any suitable iterative reconstruction technique may be used, and embodiments of the invention are not limited in this respect. An example of an iterative method, termed the algebraic reconstruction technique (ART) computes a value ρ for each of the voxels in the imaged object. A maximum likelihood estimate M² is defined as:

$${M}^{2} ( {\hat{ρ}}_{k} ) = {\sum }_{i} \frac{{( {X}_{i} ( {\hat{ρ}}_{k} ) - {x}_{i} )}^{2}}{{σ}_{i}^{2}} ,$$

where Xᵢ relates density at voxels through which a ray passes to a measured value of the ray that has passed through the object. Estimated voxel densities \hat{P}ₖ are multiplied by Xᵢ, which yields an estimate of values measured along the ith ray. By subtracting this estimate from the actual measured value xᵢ, an error value is obtained. When these error values are weighted by an uncertainty value σᵢ, squared and summed with similarly computed values along other rays, a value of M² results. The iterative technique aims to find density values ρ that minimize the changes in M² with respect to changes in density values. Density values that satisfy this criterion represent the computed image.

ART is only one many iterative reconstruction methods known in the art. Any of numerous iterative reconstruction techniques may be used instead of or in addition to ART. For instance, any of the following techniques may be used: ordered-subsets simultaneous iterative reconstruction technique (OSIRT), simultaneous algebraic reconstruction technique (SART), simultaneous iterative reconstruction technique (SIRT), multiplicative algebraic reconstruction technique (MART), simultaneous multiplicative algebraic reconstruction technique (SMART), least-squares QR method, expectation maximization (EM), ordered subsets expectation maximization (OSEM), convex method (C), and ordered-subsets convex method (OSC).

The inventors have appreciated that the use of iterative reconstruction methods allows for rapid reconstruction of images based on X-ray data collected using some embodiments of the invention. In some embodiments, an image reconstruction technique may be reconstructed using a regulator, which enables the reconstruction technique to select from among several possible image solutions. Any suitable regulator may be used, and embodiments of the invention are not limited in this respect. Illustrative regulators include, but are not limited to, a Tikhonov regulator, a total variation (TV) regulator, a Laplacian regulator, and a compressive sensing regulator.

In some embodiments, image reconstruction may be based, at least in part, on image priors that constrain the image reconstruction space. Any suitable image priors may be used, and embodiments of the invention are not limited in this respect. For example, in some embodiments image priors may be determined based, at least in part, on at least one whole-image statistics (e.g., k-means, k-nearest neighbor). In some embodiments, the at least one whole-image statistic may be based, at least in part, on one or more images of the patient from a previous imaging session. In some embodiments, image priors may be determined based, at least in part, on a plurality of images obtained from a plurality of patients, and the image priors may be used to constrain image reconstruction. For example, the plurality of images may be used to identify a set of anatomical landmarks for a particular object to be imaged (e.g., a brain, a heart, a liver, etc.), and the anatomical landmarks may be used as image priors in the image reconstruction. It should be appreciated that the plurality of images may alternatively be used in any other suitable way to determine image priors for image reconstruction in accordance with some embodiments of the invention.

An imaging system constructed in accordance with one or more of the techniques described herein may achieve an image reconstruction time for a volumetric image of acceptable image quality in substantially less time achievable using conventional CT scanners. For example, in some embodiments, a volumetric image may be reconstructed in less than one minute. In some embodiments, a volumetric image may be reconstructed in less than thirty seconds. In some embodiments, a volumetric image may be reconstructed in less than five seconds.

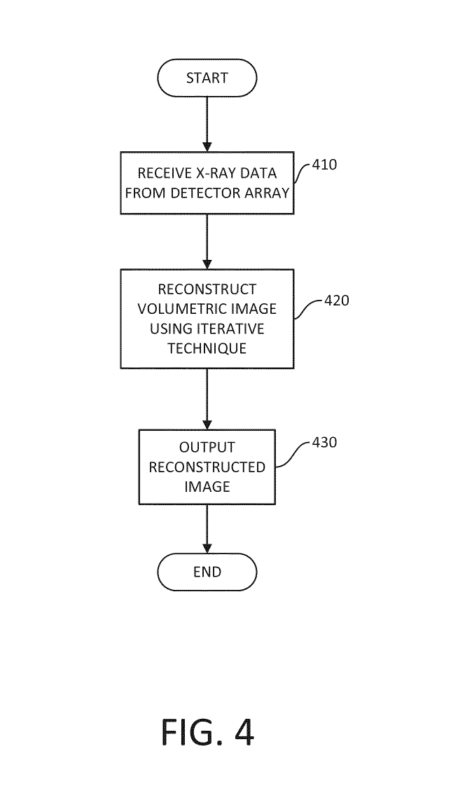

FIG. 4 shows an illustrative process for reconstructing a volumetric image based, at least in part, on X-ray data detected using some embodiments of the invention. In act 410, X-ray data is received from at least one detector array. As discussed above, the received X-ray data may correspond to data that has been collected using a range of X-ray angles substantially less than 180°, which is typically required for high-quality images taken using conventional CT scanners. The process proceeds to act 420, where the received X-ray data is used to reconstruct a volumetric image using one or more iterative reconstruction techniques, as described above. Following reconstruction, the process proceeds to act 430, where the reconstructed image is output. For example, the reconstructed image may be displayed on a screen or data describing the reconstructed image may be transmitted to another device for subsequent display and/or analysis.

Imaging apparatuses manufactured in accordance with some embodiments of the invention may include additional hardware and/or software components that facilitate use of the imaging apparatus in particular applications. For example, in some embodiments, an imaging apparatus may include a shielding structure configured to at least partially shield a person (e.g., a surgeon) other than the patient being imaged from the X-rays generated by the X-ray source. Any suitable shielding structure may be used, and embodiments of the invention are not limited in this respect. For example, a portable shielding structure may be temporarily placed between the surgeon and the patient such that the surgeon can perform medical operations on the patient during or immediately prior to irradiating the patient with X-ray radiation for imaging. Alternatively, the surgeon or other medical professional may wear a shielding structure as a protective garment or vest to shield the surgeon from X-ray radiation. Any other suitable shielding structure (including no shielding structure) may alternatively be used.

As discussed above, a limitation of some conventional CT scanners used in surgical environments is that imaging and performing medical procedures on a patient cannot be conducted simultaneously or near simultaneously because conventional CT scanners typically obstruct a physician's access to the patient while the CT scanner is positioned for imaging. Proper positioning of conventional CT scanners typically takes a substantial amount of time, which is compounded if multiple imaging sessions are necessary. Imaging apparatus in accordance with some embodiments are designed to enable a person (e.g., a surgeon) to perform at least one medical procedure on a patient placed on the table without having to move the X-ray source or the detector array. For example, by integrating the X-ray source with the table, and having a stationary detector array, only minimal (or no) changes to imaging configuration need be made to enable rapid and/or repeated imaging of a portion of a person during a medical procedures. By enabling rapid and frequent imaging sessions to be achieved, some embodiments of the invention may be better suited than conventional CT scanners in various environments including, but not limited to, military and emergency environments. For example, the use of rapid imaging may enable less or no sedation of the imaged patient and/or no movement of the patient on/off a surgical table for surgery.

As should be appreciated from the foregoing, X-ray imaging systems designed according to the principles described herein, may produce an economical, fast and accurate images for medical applications where imaging speed and portability are important or desired.

Additionally, imaging system manufactured in accordance with some embodiments of the invention may be “ruggedized” for use in harsh environments using one or more temperature-insensitive components and/or by not including moving parts that are susceptible to failure in such environments. Some embodiments may additionally or alternatively be ruggedized by sealing one or more components of the imaging apparatus to prevent foreign debris from entering portions of the apparatus, or embodiments may be ruggedized using any other suitable technique or techniques.

Alterations, modifications, and improvements are intended to be part of this disclosure, and are intended to be within the spirit and scope of the invention. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface including keyboards, and pointing devices, such as mice, touch pads, and digitizing tables. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. An apparatus, comprising:
a table on which a patient may be placed;
at least one X-ray source configured to generate X-rays at a plurality of X-ray source locations along a linear direction, wherein the at least one X-ray source is arranged to generate the X-rays such that at least some of the X-rays pass through a portion of the table in addition to passing through a portion of a patient placed on the table; and
at least one detector array comprising a plurality of detector elements and arranged to detect the at least some of the X-rays passed through the portion of the patient placed on the table, wherein the at least one detector array comprises detector elements arranged in a two-dimensional configuration.

2. (canceled)

3. The apparatus of claim 1, wherein the table includes at least one window that enables the at least some of the X-rays to pass through the at least one window.

4. (canceled)

5. (canceled)

6. The apparatus of claim 1, wherein the at least one X-ray source is mounted substantially below the table.

7. The apparatus of claim 6, wherein the at least one X-ray source includes a portion that extends at least partially above the table.

8. (canceled)

9. The apparatus of claim 1, wherein the at least one X-ray source is mounted substantially above the table.

10. The apparatus of claim 1, wherein the at least one X-ray source is configured to be stationary during a single acquisition of data used to reconstruct an image.

11. The apparatus of claim 1, wherein the at least one X-ray source is mounted such that the at least one X-ray source is translatable along a length direction and/or a width direction of the table.

12. The apparatus of claim 1, wherein the at least one source comprises at least one e-beam source.

13-15. (canceled)

16. The apparatus of claim 1, wherein the at least one source comprises a plurality of sub-sources configured to be activated using time-multiplexing.

17. The apparatus of claim 1, wherein the at least one detector array comprises a flat-panel detector array.

18. The apparatus of claim 1, wherein the at least one detector array is mounted to a movable structure.

19-21. (canceled)

22. The apparatus of claim 1, further comprising:
at least one processor programmed with instructions that, when executed by the at least one processor, reconstruct a volumetric image based, at least in part, on the X-rays detected by the at least one detector array.

23-32. (canceled)

33. The apparatus of claim 22, wherein reconstructing a volumetric image comprises reconstructing a volumetric image using information that does not satisfy a volumetric reconstruction requirement.

34. (canceled)

35. The apparatus of claim 1, further comprising:
a shielding structure configured to at least partially shield a person other than the patient from the X-rays generated by the at least one X-ray source.

36. The apparatus of claim 1, further comprising:
read-out circuitry configured to read out information from one or more detector elements of the at least one detector array, wherein the read-out circuitry is configured to provide the information read out from the one or more detector elements to at least one processor programmed to perform a volumetric image reconstruction.

37. (canceled)

38. (canceled)

39. The apparatus of claim 1, wherein the at least one source and the at least one detector are arranged to enable a person to perform at least one procedure on the patient without moving the at least one source or the at least one detector array.

40. The apparatus of claim 1, wherein the at least one source and the at least one detector are arranged to enable a person to perform at least one procedure on the patient during generation of the X-rays by the at least one X-ray source.

41. The apparatus of claim 1, further comprising:
a controller programmed to control operation of the at least one source, wherein the controller is programmed to control the operation of the at least one source to achieve less than 180 degree coverage by X-rays passing through at least one point of interest to be imaged.

42-44. (canceled)

45. A method of manufacturing an apparatus, wherein the method comprises:
integrating a table on which a patient may be placed with at least one X-ray source configured to generate X-rays at a plurality of X-ray source locations along a linear direction, wherein the at least one X-ray source is arranged to generate the X-rays such that at least some of the X-rays pass through at least a portion of the table in addition to passing through a patient placed on the table, wherein the apparatus further comprises at least one detector array comprising a plurality of detector elements and arranged to detect the at least some of the X-rays passed through the portion of the patient placed on the table, wherein the at least one detector array comprises detector elements arranged in a two-dimensional configuration.

46-49. (canceled)

50. The method of claim 45, further comprising mounting the at least one X-ray source substantially below the table.

51. The method of claim 50, further comprising positioning the at least one X-ray source such that the at least one X-ray source includes a portion that extends at least partially above the table.

52. (canceled)

53. The method of claim 45, further comprising mounting the at least one X-ray source substantially above the table.

54. The method of claim 45, wherein the at least one X-ray source is configured to be stationary during a single acquisition of data used to reconstruct an image.

55. The method of claim 45, further comprising mounting the at least one X-ray source such that the at least one X-ray source is translatable along a length direction and/or a width direction of the table.

56-79. (canceled)

80. The method of claim 45, wherein the apparatus further comprises:
read-out circuitry configured to read out information from one or more detector elements of the at least one detector array, wherein the read-out circuitry is configured to provide the information read out from the one or more detector elements to at least one processor programmed to perform a volumetric image reconstruction.

81-99. (canceled)

100. A non-transitory computer readable medium encoded with a plurality of instructions that, when executed by at least one computer processor perform a method comprising: receiving X-ray data from at least one detector array, wherein the received X-ray data does not satisfy a volumetric reconstruction requirement; and
reconstructing, with the at least one computer processor, the volumetric image using an iterative reconstruction technique based, at least in part, on the received data.

101. The non-transitory computer readable medium of claim 100, wherein reconstructing the volumetric image comprises reconstructing the volumetric image using an iterative reconstruction technique selected from the group consisting of OSIRT, SART, SIRT, OSC, C, SMART, MART, and EM.

102. The non-transitory computer readable medium of claim 100, wherein reconstructing the volumetric image comprises reconstructing the volumetric image using a regulator.

103. (canceled)

104. (canceled)

105. The non-transitory computer readable medium of claim 100, wherein reconstructing the volumetric image comprises reconstructing the volumetric image based, at least in part, on priors determined using a plurality of images from a plurality of patients and/or priors determined using at least one whole-image statistic.

106-109. (canceled)

110. The non-transitory computer readable medium of claim 100, wherein the volumetric reconstruction requirement is a requirement selected from the group consisting of a Tuy condition, a pi-line-condition, a Nyquist condition, and a non-truncation condition.
